(19) United States
(12) Patent Application Publication
(10) Pub. No.: US 2011/0082529 A1
(43) Pub. Date: Apr. 7, 2011

(54) IMPLANTABLE CONNECTION DEVICE

(75) Inventors: Fransciscus J.L. Everaerts, Eindhoven (NL); Jozef F.E. Gootzen, Eindhoven (NL); Michel Marcel Jose Decre, Eindhoven (NL)

(73) Assignee: KONINKLIJKE PHILIPS ELECTRONICS N.V., EINDHOVEN (NL)

(21) Appl. No.: 12/993,261

(22) PCT Filed: May 20, 2009
(86) PCT No.: PCT/IB09/52116

§ 371 Date: Nov. 18, 2010

(30) Foreign Application Priority Data
May 30, 2008 (EP) 08157309.9

(51) Int. Cl. A61N 1/00

(52) U.S. Cl. 607/116

(57) ABSTRACT

The invention relates to an implantable connection device (100) comprising a porous carrier (120) to which bone tissue (1) can attach and an electrical device that is embedded in the carrier or at least space for such a device. The carrier may particularly comprise ceramic particles, for example a mixture of hydroxyapatite and tricalcium phosphate. The electrical device may consist of one or more feedthrough wires to which leads (11, 21) of external devices can be coupled on both sides of the connection device. The connection device can for example be implemented into the skull (1) of a patient to provide an electrical connection between electrodes in the brain and an external pulse generator.

### FIELD OF THE INVENTION

The invention relates to an implantable connection device, particularly a device that can be implanted into the skull of a patient for providing electrical access to electrodes in the brain.

### BACKGROUND OF THE INVENTION

The WO 2005/039694 A1 discloses a method which requires the implantation of electrodes into the brain of a patient. The control and power supply to such electrodes may be achieved wirelessly, which is however a complicated, sensitive procedure with little power efficiency. A wired connection, on the contrary, has the disadvantage that the transition of the wires through the skull is subject to mechanical stress which may lead to wire breaking Moreover, the feedthroughs of the wires are often subject to inflammations.

### SUMMARY OF THE INVENTION

Based on this situation it was an object of the present invention to provide means for an electrically reliable and physiologically well compatible access to electronic components through a bone structure.

This object is achieved by an implantable connection device according to claim 1. Preferred embodiments are disclosed in the dependent claims.

The device proposed by the present invention will be called “implantable connection device” in the following because it shall be implantable into the living body of a human or animal patient (i.e. it must be small enough for this purpose and biocompatible) and because it shall serve for the electrical connection of different components, particularly components located at opposite sides of a bone structure like the skull. The implantable connection device comprises the following components:

a porous carrier to which bone tissue can attach, wherein said attachment may comprise a contact limited to the surface of the carrier and/or a growth of bone tissue into the carrier.

an electrical device that is embedded in the carrier or at least a space (cavity) in the carrier reserved for such an electrical device. The device may for example be a pulse generator for delivering electrical pulses to electrodes located in adjacent tissue.

The described implantable connection device has the advantage to provide a protected, stable, and well defined seat for an electrical device, as this is embedded in a carrier during the (industrial) fabrication of the connection device or during its later application. Additionally, the connection device is prepared to be optimally integrated into bone tissue due to its porous carrier to which said tissue can attach. Thus the whole system can (after healing) be intimately integrated into the body of a patient while the sensitive electrical components are kept in a secure, protected artificial structure.

The carrier has preferably an open structure, i.e. the pores of the carrier are connected to form connected paths through the material. In this case it is possible that (bone) tissue can grow into the carrier and thus integrate it into the body.

The carrier may optionally comprise a degradable and a non-degradable material, preferably in the form of a homogeneous or heterogeneous mixture. A degradable material can step-by-step be replaced by body tissue during the healing process, thus allowing an optimal integration of the connection device into e.g. the skull of a patient. By choosing the ratio of degradable and non-degradable materials appropriately, the speed and the final result of this integration can be controlled as desired. Moreover, it is possible to produce a connection device with a spatially varying composition ratio, having for example a higher fraction of non-degradable material in the vicinity of the electrical device and a higher fraction of degradable material in its periphery.

The carrier may preferably comprise a ceramic material as this combines a bone-like stability with good biocompatibility.

The aforementioned ceramic material may optionally be organized in a random or a regular matrix structure of ceramic particles (wherein said particles may themselves be of random or regular shape). Thus physical properties like density or porosity can be set as desired by an appropriate choice of the structure.

The ceramic material may preferably comprise hydroxyapatite and/or tricalcium phosphate, which are a non-degradable and a degradable material, respectively, with good compatibility to bones.

The integration of the implantable connection device into a bone tissue can further be enhanced if the carrier comprises bone chips, collagen, blood clots and/or bone forming promoting drugs. By choosing the combination and amount of these materials, it is possible to take influence on the integration process of the connection device into the body according to the individual requirements of each case (size of the connection device, location in the body, age of the patient etc.).

The carrier may further comprise an electrically conductive material and/or particles with an electrically conductive coating. In this way it is possible to prepare the carrier for the flow of electrical signals and/or power.

The electrical device that is (or that can be) embedded in the carrier may be any kind of electric or electronic component to be implanted near or into bone tissue. In a case with great practical importance, the electrical device comprises or consist of at least one electrically conductive wire or lead that electrically connects opposite sides of the connection device. In this case the implantable connection device can provide electrical access through bone material, for example access to an interior cavity of the bone (e.g. the skull). An advantage of such a wired connection is that it is highly reliable, robust, and energy efficient. Moreover, the wire is at the same time optimally integrated into the bone tissue and firmly embedded in the carrier. The wire may for example be made of a metal (copper, gold etc.) or a conductive polymer.

The aforementioned wire(s) will typically be connected to further devices on both sides of the connection device, for example to an implemented stimulation electrode on a first (interior) side and a pulse generator on a second (exterior) side. If possible, the wire may run in one piece to these further devices. In most cases, it will however be necessary to connect the wire which is embedded in the carrier to other electronic terminals, for example to wires leading to the mentioned stimulation electrode or pulse generator. Such a connection of the embedded wire to an electrical terminal can be done by any appropriate means, for example by soldering, welding, gluing, crimping or the like. Preferably, the wire embedded into the connection device is (reversibly or permanently) coupled to at least one connector to which an external electrical terminal can (reversibly or permanently) be coupled, too. Thus a comfortable plug-and-socket type connection can be established, which is very easy to connect during a surgical intervention and which allows an exchange of electrical components without a removal of the implanted connection device.

The wire(s) mentioned above may follow a more or less random path through the carrier. Alternatively, they may run through a tubular space in the carrier, which provides a short path with minimal bending stress for the wires and, most of all, the possibility to insert the wires later (e.g. during a surgical intervention) into the carrier.

The wire(s) running through the carrier may optionally be attached to particles of the carrier material (for example in a process like sintering). Thus a firm connection between the wire and the carrier material is provided.

### BRIEF DESCRIPTION OF THE DRAWINGS

These and other aspects of the invention will be apparent from and elucidated with reference to the embodiment(s) described hereinafter. These embodiments will be described by way of example with the help of the accompanying drawings in which:

FIG. 1 shows in a schematic sectional view a first implantable connection device according to the present invention implanted into the skull of a patient;

FIG. 2 shows the first implantable connection device separately in a perspective view;

FIG. 3 shows an enlarged view of two wires running through an irregular carrier of a second implantable connection device according to the present invention;

FIG. 4 shows an enlarged view of two wires running through a regular carrier of a third implantable connection device to the present invention;

FIG. 5 shows a schematic perspective view of a fourth implantable connection device to the present invention with parallel conductive paths.

Like reference numbers or numbers differing by integer multiples of 100 refer in the Figures to identical or similar components.

### DETAILED DESCRIPTION OF EMBODIMENTS

Several surgical procedures are described in literature where electrodes are implanted “in the brain” and electrical pulses are delivered to specific locations to treat a disease. Examples of these diseases are Parkinson's disease and a variety of clinical depressions. Also stimulation of the cochlear area is used to support patients with hearing disorders. In all of these cases an electrode is (or multiple electrodes are) implanted within the skull area. The tip of this electrode is the delivery vehicle and transfers electrical pulses to the surrounding tissue. The electrode (tip) is connected in one or the other way to a device generating these electrical pulses.

The aforementioned electrode is usually implanted during the surgical procedure of a craniotomy. In most cases a wire connected to the electrode at one side is fed through the skull to an external device located beneath the skin (typically subcutaneous under the clavicle, sub-muscular under the clavicle or subfascial), and the required wiring is positioned just below the skin. After replacement of the bone flap the electrode remains.

In general, there are several options about how electrical pulses may reach the electrode tips. One method is the aforementioned use of conducting paths (“wires” or “leads”) from the device to the electrode tips. In “wireless” options, energy and/or communication is transferred through the skull bone by using electrical fields, magnetic fields, light, acoustic field or combinations. In the wireless method no wire has to be fed through the bone material, which is advantageous as such a feedthrough can be a source of inflammation or a cause of mechanical failure of the wires by fatigue. However the wireless technology has also some drawbacks since it is energy inefficient and may cause an undesirable local heat production.

To address the above issues, the present invention proposes to use an implantable connection device (or “interconnect”), embedded in the skull bone where at one side an electrical device like a pulse generator or wires from such a device are connected (“outer wires”) and at the other side wires are attached and connected to the electrode tips (“inner wires”). This approach is beneficial since an optimal interconnect design, that totally is embedded in the bone, completely separates the two environments at both sides of the skull.

As brain tissue is different from tissue in the subdermal pocket, events as healing, inflammation, or scar tissue formation have different origin and proceed via different biological pathways. The proposed implantable connection device allows in this respect for the optimization of material selection and mechanical properties for both inner and outer wires. In addition, attachment of drugs or smart molecules (biological or synthetic) to the inner or outer wires may improve performance. Some of these molecules may for example be very beneficial if used subdermally while they are not desired in combination with brain tissue.

By using the proposed implantable connection device, replacement of outer wires becomes relative easy since the device can be accessed by only a small incision in the skin. Moreover, a patient could thus readily be connected to an external or therapeutic device located in a neurological suite for e.g. fine tuning or mapping purposes.

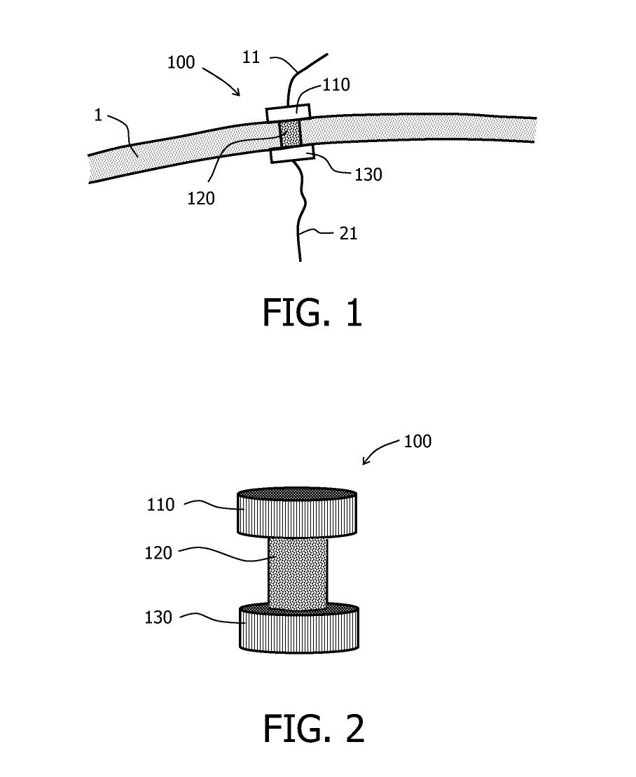

FIG. 1 shows schematically a section through the skull 1 of a patient, wherein a first embodiment of an implantable connection device or “interconnect” 100 according to the present invention is implanted into a hole in the skull. An outer wire 11 leading to an external device like a pulse generator (not shown) is coupled to a connector 110 of the connection device 100 that is located on the outside of the skull 1. Similarly, and inner wire 21 leading to an electrode in the brain tissue (not shown) is attached to an interior connector 130 of the connection device 100. The interior and outer connectors 110 and 130 are mechanically coupled by a porous carrier 120 that is embedded into the skull 1. They are also electrically coupled by a (single) wire (not shown) embedded into the carrier 120 and/or by making the whole carrier 120 electrically conductive.

FIG. 2 shows a perspective view of the implantable connection device 100 of FIG. 1. It can be seen that this device 100 is composed of three cylindrical elements, namely the exterior connector 110, the carrier 120, and the interior connector 130.

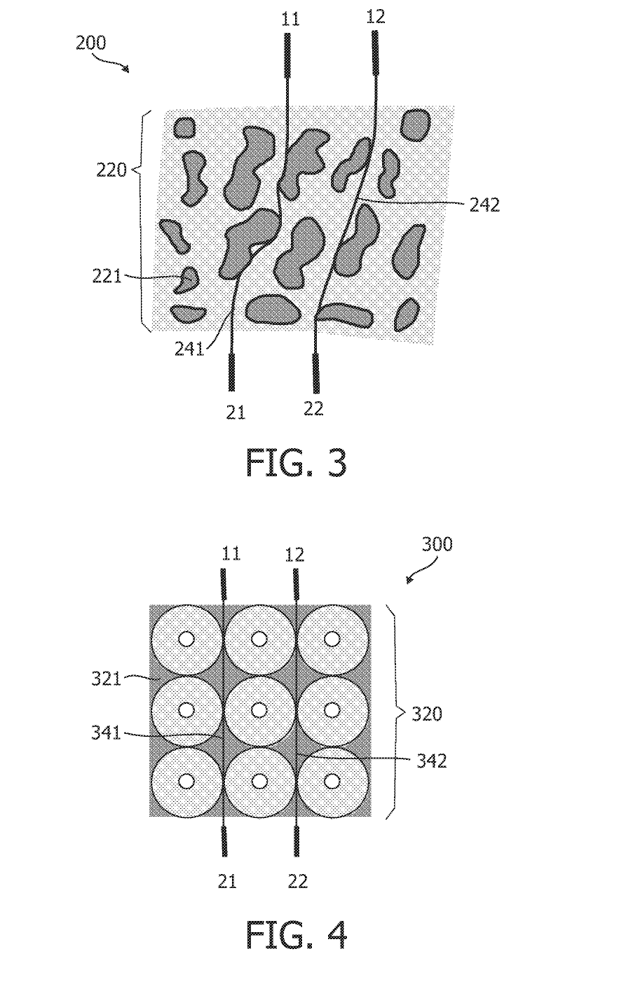

FIG. 3 shows the interior structure of the carrier 220 of a second implantable connection device 200 according to the present invention. The carrier comprises a matrix of randomly shaped and located ceramic particles 221 forming an open pore structure between them. Two internal feedthrough wires 241 and 242 run (roughly parallel to each other) through the empty connected spaces of the carrier 220 and finally connect to external wires 11, 12, and internal wires 21, 22, respectively. In general, the electrically connecting pathways may be made of e.g. a metal conductor or a conducting polymer.

FIG. 4 shows in a representation similar to FIG. 3 the internal structure of the carrier 320 of a third implantable connection device 300. In this case ceramic particles 321 are regularly shaped and positioned in a regular (periodic) matrix.

The well organized particles with their pre-defined interpores and holes allow for an optimal bone formation. Internal feedthrough wires 341, 342 run through tubular spaces of the carrier, being attached to the ceramic particles 321.

The connecting pathways (wires) may be incorporated into the ceramic carrier during production. Alternatively, the ceramic material may contain hollow, tube shaped openings. During application, an electrode wire can then be fed trough these openings by the surgeon and become incorporated in the newly formed bone. In this case (temporarily) fixation of the electrode wires may be required.

The carrier of the described implantable connection devices may optionally comprise a conducting material or conducting materials (metals or ceramics with conductive coatings). In this way a conductive path leading through the connection device can be created which may replace an internal wire of the kind described above. If the regions of the carrier that comprise conductive materials are separated from each other by an isolating material, it is even possible to construct multiple connection pathways through the carrier.

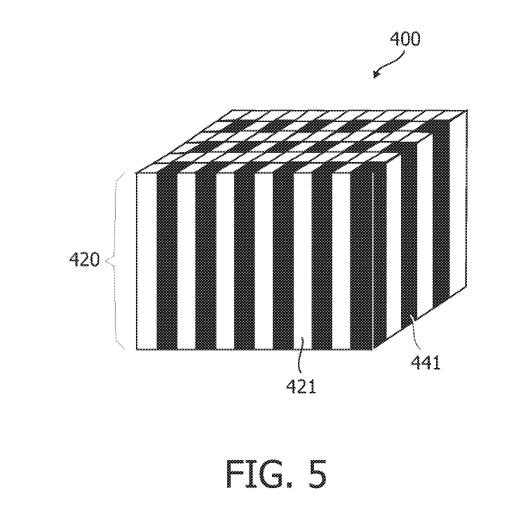

FIG. 5 shows in this respect the carrier 420 of a fourth implantable connection device 400. The carrier 420 comprises a plurality of parallel conductive paths 441 separated by isolating ceramic material 421. Such a structure may for example be produced by carefully ordering metal coated polymer beads and bare polymer beads such that the conducting pathways are made with an insulation layer in between. To this end a mandrel can for example be used that has a plurality of parallel compartments, wherein each compartment is filled with metal coated beads (black in FIG. 5) or non-coated beads (white). After removal of the mandrel, a 3D polymer mask is created consisting of several conducting pathways 441 separated by isolators 421. After filling said polymer mask with a ceramic slurry, burning of the polymer beads, and formation of the ceramics, the final shape is obtained.

The carrier 120 of the connection device 100 of FIGS. 1 and 2 could be structured like the carriers 220, 320 or 420 of FIGS. 3 to 5, respectively (besides the fact that in these embodiments several internal feedthrough wires are used instead of one).

An open structure of the carrier is usually preferred as this will promote bone formation and therefore fusing of the connection device with the natural bone.

Attachments of the interior and exterior wires 11, 12, 21, 22 can be made by crimping, gluing, soldering or welding (ex vivo). Also additional “standard’ connectors 110, 130 as indicated in FIGS. 1 and 2 can be used to allow for easy and reversible “plug-in” connections.

The carrier preferably comprises a (bio) ceramic material with particles made for example of hydroxyapatite (HA, non-degradable) and/or tricalcium phosphate (TCP, degradable). The ceramic material can for instance be made of 100% HA, or it can consist of a combination of HA and TCP. The ratio of HA and TCP will then determine the degradation of this “artificial bone”, wherein a ratio HA/TCP of about 60/40 may be used as “golden standard”; however, other ratios may be used as well. Further details with respect to suitable carrier materials can be found for example in the U.S. Pat. No. ,4195,366, U.S. Pat. No. 4,629,464, U.S. Pat. No. 5,017,518, U.S. Pat. No. 5,096,814, U.S. Pat. No. 5,266,248, U.S. Pat. No. 5,355,898, U.S. Pat. No. 5,531,794, U.S. Pat. No. 5,549,123, U.S. Pat. No. 5,916,553, or U.S. Pat. No. 6,149,688.

There are numerous methods to produce a porous ceramic material. A porous ceramic material with an open, well organized structure containing interporous connections can for example be produced by using polymer beads (such as PMMA) to make a negative impression of the ceramic material. In case of the method described in this application, metal wires or other conducting materials are placed in a mold that is thereafter filled with the polymer beads. The mold is placed in a oven and heated until the glass transition temperature of the polymer beads is reached. The oven conditions and time of exposure are controlled such that the beads melt and attach together. Then a ceramic slurry is poured in the mold and the remaining openings between the beads are filled. After drying, the mold containing this green shape is placed in a sintering oven, and in two heating cycles the ceramic material is formed. The aim of the first step is to remove the polymer while in the second step the ceramic is sintered. More details on methods for the production of ceramic materials can for example be found in the U.S. Pat. No. 6,037,519, U.S. Pat. No. 6,346,123, and US20060257449.

The carrier is placed during a surgical intervention such that the area containing the mesh or pores is at the level of the skull bone. If the opening in the skull is small enough, non-critical spontaneous healing and closure of the wound will occur. If the opening is critical, the open structure of the carrier may optionally be filled with bone chips, collagen, blood clots with or without addition of a bone forming promoting drug (such as BMP-2).

The implanted connection device has to be kept in place until the healing process has been finished. This can be done by temporarily but also permanent fixation to the skull. Small screws but also glue or other methods known to a person skilled in the art can be used for this.

Once in place, the connection device will separate the outer from the inner environment and provide direct electrical access to the electrodes located inside the skull. If healing has occurred, the connection device completely separates the outer from the inner area of the skull.

It should be noted that more elaborate electrical devices than just feedthrough wires could be embedded in the ceramic carrier of the proposed implantable connection device.

The implantable connection devices described above may particularly (but not exclusively) be used in the skull area, for instance in connection with deep brain stimulation or the treatment of hearing disorders. The devices allow for example to create a wired connection between a cochlear implant and electronics located outside the skull.

Finally it is pointed out that in the present application the term “comprising” does not exclude other elements or steps, that “a” or “an” does not exclude a plurality, and that a single processor or other unit may fulfill the functions of several means. The invention resides in each and every novel characteristic feature and each and every combination of characteristic features. Moreover, reference signs in the claims shall not be construed as limiting their scope.

1. An implantable connection device (100-300), comprising
a) a porous carrier (120-320) to which bone tissue (1) can attach;
b) an electrical device (241, 242, 341, 342) that is embedded in the carrier and/or space for such an electrical device.

2. The implantable connection device (100-300) according to claim 1,
characterized in that the carrier (120-320) has an open structure.

3. The implantable connection device (100-300) according to claim 1,
characterized in that the carrier (120-320) comprises a degradable and a non-degradable material.

4. The implantable connection device (100-300) according to claim 1,
characterized in that the carrier (120-320) comprises a ceramic material (221, 321).

5. The implantable connection device (100-300) according to claim 4,
characterized in that the ceramic material comprises a random or a regular structure of ceramic particles (221, 321).

6. The implantable connection device (100-300) according to claim 4,
characterized in that the ceramic material comprises hydroxyapatite and/or tricalcium phosphate.

7. The implantable connection device (100-300) according to claim 1,
characterized in that the carrier (120-320) comprises bone chips, collagen, blood clots, and/or a bone forming promoting drug.

8. The implantable connection device (100-300) according to claim 1,
characterized in that the carrier (120-320) comprises a conductive material and/or particles with a conductive coating.

9. The implantable connection device (100-300) according to claim 1,
characterized in that the electrical device comprises at least one wire (241, 242, 341, 342) that electrically connects opposite sides of the connection device.

10. The implantable connection device (100-300) according to claim 9,
characterized in that the wire is coupled at at least one end to a connector (110, 120) to which external leads (11, 21) can be coupled.

11. The implantable connection device (300) according to claim 9,
characterized in that the wire (341, 342) runs through a tubular space in the carrier (320).

12. The implantable connection device (100-300) according to claim 9,
characterized in that the wire (241, 242, 341, 342) is attached to particles (221, 321) of the carrier.

13. The implantable connection device (100-300) according to claim 9,
characterized in that the wire (241, 242, 341, 342) is made of a metal or a conductive polymer.
